(19) United States
(12) Patent Application Publication
(10) Pub. No.: US 2005/0112180 A1
(43) Pub. Date: May 26, 2005

(54) Antimicrobial elastomeric flexible article and manufacturing method

(76) Inventor: Belle L. Chou, Union City, CA (US)

Correspondence Address: Otto O. Lee;Intellectual Property Law Group LLP, Suite 1205, 12 South First Street, San Jose, CA, 95113, US

(21) Appl. No.: 10/719,573

(22) Filed: Nov. 22, 2003

(51) Int. Cl. A61K 9/70

(52) U.S. Cl. 424/443; 442/123

(57) ABSTRACT

An elastomeric flexible article and method for manufacture has antimicrobial characteristics. An example of such an article is a disposable protective glove that includes a first layer, with an effective amount of antimicrobial agent therein or thereon; and a second layer, to be closer to a hand than the first layer, when the glove is worn on the hand. The second layer is configured to resist, when the glove is worn, penetration by the anti-microbial agent and thereby to resist contact between the anti-microbial agent with the hand.

### BACKGROUND

The present invention relates to antimicrobial elastomeric flexible articles, for example, gloves or the like.

Disposable gloves, for example, have played a significant role in the fields of chemistry, biology, and medicine by being widely used as a protective measure to insulate hands from objects handled by a glove wearer. Disposable gloves have been widely used within the food industry, in which gloves are commonly used to protect against food contamination during food preparation, and within the medical community, in which gloves have been worn by health care professionals such as surgeons, nurses, dentists and other personnel for protection from infectious agents. The medical community has long been concerned about microbial cross-contamination between patients and health care professionals. Health care professionals frequently wear gloves as a physical barrier form of protection to reduce the risk of being exposed or contaminated through hands by infectious agents such as viruses or bacteria.

To allow ease in handling objects, conventional disposable gloves typically are made of thin and elastic materials to minimize the space between the skin and the glove. One disadvantage with this type of glove is that it has been suggested that channels can exist in, for example, latex gloves, which can allow viruses to pass through to the user's hand. Although it is customary for health care professionals to wash their hands frequently with an antimicrobial agent in a skin cleanser before donning gloves, the effect of the antimicrobial agent may be short-lived and the infectious agents such as viruses or bacteria may regrow beneath the gloves in the moist warm environment. A further disadvantage is that prolonged wearing of disposable gloves can cause a moist environment on the surface of the hand that allows viruses, bacteria, yeast, fungus and other infectious agents to grow and multiply. Itchiness and irritation can be a frequent result of wearing disposable examination gloves for extended periods.

An additional problem with disposable gloves commonly found among, for example, health care professionals, are applications which can expose the outside of the glove to infectious agents such as microbes from patients or work surfaces or any other object with which the gloves are in contact. These infectious agents can remain on the glove and infect, for example, a patient via cross-contamination or the glove wearer. Also, there have been gloves such as conventional polyvinyl chloride gloves that can be dusted with an antimicrobial agent, but the agent would not be effective for prolonged periods of use because the antimicrobial agent would rub off or disappear completely when the gloves are immersed in water. To alleviate perspiration, powders are commonly used on the inner surface of disposable gloves, in addition to making donning, wearing, and removal of gloves easier. However, there are several disadvantages that can be associated with powders. Continuous perspiration can easily overwhelm the thin layer of powder that is commonly on the surface of the glove. This is especially the case when continuous and frequent wearing of gloves is required. For example, dentists may continuously wear gloves during a dental surgical procedure for up to 40 minutes or more. In addition, hand washing is necessary after the use of powdered gloves. Frequent hand washing to remove powders is inconvenient and may also cause excessive dryness of the skin.

Still further, conventional skin preparations for gloves may be incapable of prolonged effectiveness within gloves, in the presence of accumulating perspiration and other substances that can overwhelm the preparations. Still further, conventional skin preparations for gloves may contain substances that are undesirable to some users for some applications, for example, substances that are unfamiliar to users (for example, antibacterial agents that do not occur naturally) or substances that are suspected of being harmful (for example, conventional antiperspirants).

### SUMMARY

The present invention is directed toward improved elastomeric flexible articles, for example, disposable examination gloves or the like.

According to an embodiment of the present invention, there is a disposable protective glove comprising a first layer, with an effective amount of antimicrobial agent therein or thereon; and a second layer, to be closer to a hand than the first layer, when the glove is worn on the hand, the second layer configured to resist, when the glove is worn, penetration by the anti-microbial agent and thereby to resist contact between the anti-microbial agent with the hand.

According to another embodiment of the present invention, there is a disposable protective article comprising an outer layer having an antimicrobial agent distributed within or applied onto the outer layer; and an inner layer to be closer to the skin than the outer layer, the inner layer having less proteins than natural rubber latex and comprising an interior surface with a skin conditioning or soothing substance dispersed thereon; wherein the inner layer serves as a barrier between the skin and the outer layer so to resist developing of antimicrobial resistance in microbes on the skin and some of the skin conditioning or soothing substance will interact physically with perspiration from the skin and thereto increase in ability to condition or soothe the skin.

According to another embodiment of the present invention, there is a method for making a disposable protective article for protecting skin, the disposable protective article to comprise multiple layers. The method comprises forming a first layer, the first layer comprising a material that includes an antimicrobial agent dispersed within; and forming a second layer, the second layer to be closer to the skin than the first layer when the disposable protective article is in use, wherein the second layer is to help resist contact between skin and the antimicrobial agent when the disposable protective article is used on skin.

According to other embodiments of the present invention, there is a method for making any glove according to any embodiment of the present invention.

According to other embodiments of the present invention, there is a glove made by a method according to any embodiment of the present invention.

### DESCRIPTION OF THE DRAWINGS

Features, aspects, and advantages of some embodiments of the present invention will become better understood with reference to the accompanying drawings, which are not to be considered limitative in the scope of the invention, but are merely illustrative.

FIG. 1 shows a front perspective view of one embodiment of the present invention, in which the antimicrobial elastomeric flexible article is a glove.

FIG. 2 is a sectional view of the antimicrobial elastomeric flexible article shown in FIG. 1.

### DETAILED DESCRIPTION OF SPECIFIC EMBODIMENTS

The drawings and the description in the present document, including the abstract, describe one or more currently preferred embodiments of the present invention and also describe some optional features and alternative embodiments. The description and drawings are for the purpose of illustration and not limitation. The title, section titles, and the like of the present document are terse and are for convenience and not limitation.

According to an embodiment of the present invention, there is an antimicrobial elastomeric flexible article. According to another embodiment of the present invention, there is a method of manufacturing.

The following references are hereby incorporated by reference in their entireties for all purposes:

U.S. patent application Ser. No. 10/138,370, filed May 2, 2002, entitled “An Elastomeric Flexible Article And Manufacturing Method”;

U.S. Pat. No. 6,423,328, entitled “Aloe Vera Glove and Manufacturing Method”; and

U.S. Pat. No. 6,630,152, also entitled “Aloe Vera Glove and Manufacturing Method”.

First and Second Layers

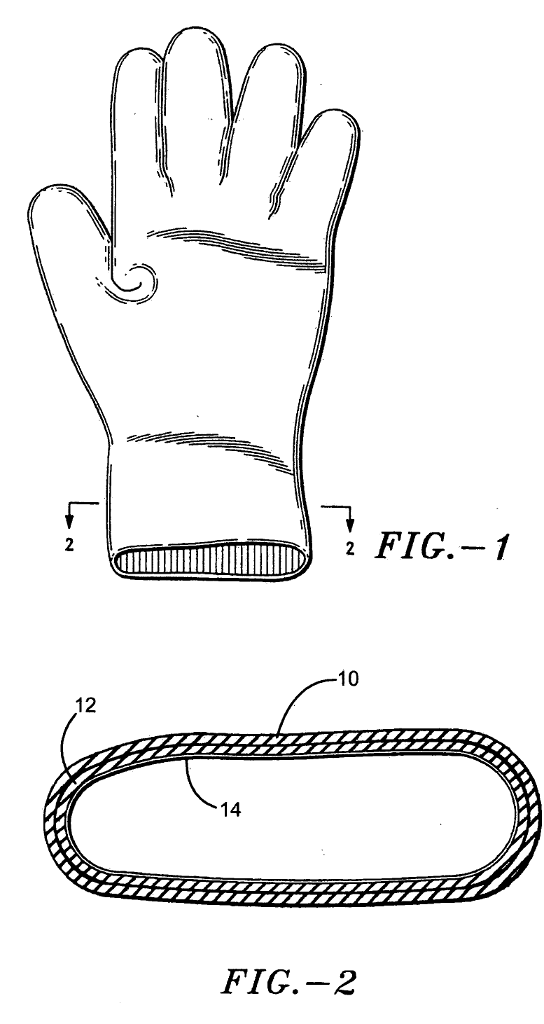

As illustrated in FIGS. 1 and 2, an antimicrobial elastomeric flexible article according to some embodiments of the present invention is a disposable protective glove. The disposable protective glove includes a first layer 10 and a second layer 12. The flexible article is shown as a glove in FIGS. 1 and 2, but other forms of articles may also be used, for example, condoms, or other protective articles or the like to be worn on, or to cover, a portion of the body, or the like. For example, the protective glove may be embodied as a glove without any layer of porous material overlying the hand, e.g., without any layer of porous material.

During use, the first layer 10 includes an effective amount of antimicrobial agent dispersed within or coated on an outer surface of the first layer 10. The effective amount of antimicrobial agent is capable of inhibiting proliferation of infectious agent(s) that comes into contact with first layer 10. The second layer 12 is closer to a user's hand than the first layer. When the glove is worn on the user's hand, the second layer 12 can be sufficiently configured to resist penetration by the anti-microbial agent from the first layer 10 and to resist contact by the anti-microbial agent from the first layer 10 with the hand. The second layer 12 can provide an additional barrier to protect the user from infectious agents.

In some embodiments of the present invention, the elastomeric flexible article is a protective glove that is simple and convenient to use and allows the user to wear the glove and to perform fine tasks with precision. For example, the glove may be embodied as a disposable examination glove made of at least two layers, a first layer 10 and a second layer 12. The first layer 10 can be made of a single layer that can be made of various materials known to those of ordinary skill in the art. Resinous materials such as vinyl or the like or polymer materials such as acrylonitrile or the like are common choices. Three commonly used materials for making disposable gloves are natural rubber latex, acrylonitrile, and polyvinyl chloride, although any other elastomeric material may also be used. Still other materials, for example, polyurethane, chloroprene, neoprene, butadiene, or the like, or any elastomeric material known to those with ordinary skill in the art may also be used.

The second layer 12, which is closer to the user's hand, can be made of a single layer of fluid-impermeable material that can provide an additional barrier to help protect the user from substances associated with the first layer 10 (e.g., allergenic proteins or antimicrobial agents) and from infectious agents that may have penetrated the first layer 10. For example, second layer 12 may be embodied to include no layer of porous material overlying the hand, e.g., without any layer of porous material. The second layer 12 can include, for example, any of the above materials used for the first layer 10 that are fluid impermeable. Preferably, the second layer 12 is not made of latex since latex can have allergenic proteins that are difficult or expensive to satisfactorily remove or deal with. Thus, preferably, the second layer 12 includes less proteins than natural latex. In addition to the specific materials discussed, any combination of suitable materials may be used, e.g., nitrile-nitrile, or any other combination.

In some embodiments of the present invention, the elastomeric flexible article is a protective glove that has an overall thickness at the fingers of no more than about 0.3 mm, or no more than about 0.2 mm. The glove may have a minimum thickness at the fingers of at least about 0.08 mm. The glove may include two layers as discussed in the present document, with the inner layer having, at the fingers, at least about 5 percent, or at least about 10 percent of the thickness of the glove. Still, other thicknesses can be chosen, depending on the intended application.

Further Discussion of First Layer

In accordance with some embodiments of the present invention, the first layer 10 typically includes an effective amount of antimicrobial agent, dispersed within first layer 10 or disposed onto, or adjacent to, or overlying a surface of first layer 10. The first layer 10 preferably is the outer layer of the elastomeric article that may be exposed to an infectious patient or work surface. An effective amount of antimicrobial agent is the least sufficient concentration to prevent, decrease, or inhibit the growth and proliferation of infectious agents such as bacteria, viruses, and fungi, or the like, during at least some period of use of the elastomeric article. The first layer 10 typically can provide a protective barrier to infectious agents during a period of glove use and diminish cross contamination.

To disperse the antimicrobial agent within first layer 10, any competent method can be used. For example, the antimicrobial agent may be incorporated into a composition (e.g., a liquid or slurry) that will coagulate to form the first layer 10. The antimicrobial agent can be dispersed substantially homogeneously throughout the first layer of the glove, e.g., by mixing the composition to which the antimicrobial agent has been added.

The dispersement of the antimicrobial agent into the first layer 10 can result in the release of some of the antimicrobial agent from either surface of the first layer during use, resulting in a glove that is effective in reducing (preferably significantly) or inhibiting (preferably substantially) infectious agents on the glove's surface and throughout the first layer 10. Additionally, the dispersement of the antimicrobial agent in the first layer can provide continuous effectiveness during a period of use, by continuing to release the antimicrobial agent from its surface(s) over time. The particular period of use depends on the application, and can be any period of use. For example, at least ten minutes, or at least 30 minutes, are some possible periods of use. Many other periods of use are possible.

To dispose the antimicrobial agent onto or adjacent to a surface of first layer 10, any competent method can be used. For example, the antimicrobial agent can be dispersed on a surface of the first layer, preferably on the outer surface, e.g., by being spray-coated or immersion-coated onto the formed or forming first layer of the glove.

Embodiments of the present invention can use any competent antimicrobial agent. Some examples of such agents include halogenated hydroxy diphenyl derivatives such as diphenyl ethers, phenol derivatives, diacetylamino-azotoluene and triclorocarban, 2,4,4′trichloro-2′-hydroxydiphenyl ether (triclosan), chlorophene, and dichloroxylenol, hexachlorophene, or the like. Other agents can also be used.

A currently preferred embodiment of the present invention is a glove wherein the effective antimicrobial agent is 2,4,4′trichloro-2′-hydroxydiphenyl ether (triclosan). Triclosan is a broad-spectrum antimicrobial agent that is commercially available under the name Microban.™. (Clinitex Corp.)

According to some embodiments of the present invention, the antimicrobial agent may be present from about 0.1% by weight to about 10% by weight of the total dry weight of the first layer of the glove. Preferably, the antimicrobial agent is present from about from 0.1% by weight to 5% by weight of the total dry weight of the first layer of the glove. More preferably, the antimicrobial agent is present from about 0.3% by weight to 3% by weight of the total dry weight of the first layer of the glove. Still other amounts of antimicrobial agents may be used. Further, the amounts chosen to be used can depend on the type of agent used. It is believed that a glove with sufficient antimicrobial agent, for example, more than about 4% antimicrobial agent can have not only an effective antimicrobial effect on a surface of the glove but also an antimicrobial effect on surfaces contacted by the glove. Thus, transfer of harmful infectious agents from the glove to the surface contacted could be significantly reduced or even (for some period of use) substantially eliminated.

According to a currently preferred embodiment, the antimicrobial agent is triclosan, and the antimicrobial agent can be dispersed throughout the first layer, the amount of the antimicrobial agent may be present less than about 1% by weight of the first layer. If the antimicrobial reagent is triclosan and is sprayed or applied by immersion to form a coating on the outside of the first layer, the amount of triclosan, according to an embodiment of the invention, may be present from about 3% by weight to about 5% by weight. Still other concentrations are possible.

Further Discussion of Second Layer

Some embodiments of the present invention include a second layer 12 that can provide an additional barrier to protect the user from infectious agents. According to some embodiments of the present invention, the second layer 12 of the disposable protective glove includes an interior surface and a preparation 14 disposed on the interior surface. In accordance to another embodiment of the present invention, the second layer 12, can provide beneficial results to the user's hands. The interior surface of the second layer 12, is closer to a user's hand than the first layer. When the glove is worn on the user's hand, the second layer 12 can be sufficiently configured to resist penetration and contact by the anti-microbial agent from the first layer 10 with the hand. In accordance with some embodiments of the present invention, the second layer 12 and first layer 10 are laminated together, e.g., the surfaces are in direct contact with one another.

In accordance with some embodiments of the present invention, preparation 14 can further include an additional anti-microbial substance. In one embodiment, the preparation 14 uses an anti-microbial substance that is a naturally-occurring substance. For example, the anti-microbial substance in the preparation 14 may be a plant-derived, or edible-plant-derived, acid, and the preparation 14 may include a buffer that helps resist change in pH during wearing of the disposable protective glove. The interior surface of the second layer 12 coated with preparation 14, preferably is in contact with the user's skin, which can intermingle with perspiration from the skin, and due to the presence of the preparation 14, has a property of being antibacterial, antiviral, or a combination thereof. Additional antimicrobial substances can overcome microbial growth that can form when a user's hand perspires inside a glove. For example, an embodiment of the preparation 14 may be any embodiment described in the incorporated-by-reference U.S. patent application Ser. No. 10/138,370. In yet another embodiment of the present invention, the preparation 14 can include moisturizers and/or soothing agents such as aloe vera which can provide beneficial results to the user's hand. For example, the preparation 14 may include ingredients from any coating described in the incorporated-by-reference U.S. Pat. No. 6,423,328 or 6,630,152, for example, aloe vera or other skin-beneficial substances. Generally, the preparation 14 may embody any coating described in the incorporated-by-reference U.S. Pat. No. 6,423,328 or 6,630,152 or U.S. patent application Ser. No. 10/138,370, or any coating that is a combination of any of the ingredients for coatings described therein. For example, the preparation may include both an antimicrobial agent (e.g., a naturally-occurring type of acid, e.g., with an additional buffer) and also a skin-benefiting substance (e.g., aloe vera).

Methods of making various embodiments of preparation 14 are as set forth in the incorporated-by-reference U.S. patent application Ser. No. 10/138,370, U.S. Pat. Nos. 6,423,328, 6,630,152, or any combination thereof.

According to one embodiment of the present invention, during use of the glove, the environment encountered by the hand within the glove is acidic, due to presence of the preparation 14. The acidic environment not only can provide an extra layer of microbial protection for the user, but can also provide beneficial results by exfoliating and smoothing a user's hands. For example, the preparation may be an acidic preparation that has been dried onto the inner surface of the glove, and perspiration from the hand moistens the dried acidic preparation. For example, no other moisture is introduced into the worn glove, other than by perspiration. The acidic preparation may be a mixture that includes an acidic solution, and the mixture may, but need not, itself be a solution. Preferably, the preparation 14 contains a buffer, to help maintain the pH and stabilize pH drift. Whether or not the preparation was dried onto the inner surface of the glove, the preparation during use is acidic in the embodiment. Preferably, the pH of the preparation 14 during use is lower than about 6, for example, between about 3.8 to about 6, or, more preferably, between about 4.5 to about 6, or between about 5 to about 5.8. Preferably, the preparation 14 is formulated to maintain pH within the desired range even after some prolonged use, e.g., even after some prolonged perspiration. Low pH may be used to provide skin exfoliation.

The preparation 14 may be disposed onto the interior surface of the second layer 12 by any manner whatsoever. For example, the preparation may be disposed onto the elastomeric flexible article in dry (e.g., powder) or moist (e.g., wet mixture) form. In one embodiment of the present invention, the preparation is preferably disposed onto the elastomeric flexible article, e.g., glove, in non-powder form. Preferably, the preparation is disposed onto the elastomeric flexible article in non-dry form and then is preferably fully or at least substantially dehydrated. Preferably, the dehydration is conducted such that the preparation is dehydrated onto the elastomeric flexible article, and such that there is a force provided by the dehydration that attaches the preparation to a surface of the elastomeric flexible article. Preferably, the preparation is disposed onto the elastomeric flexible article during factory production, and not by an end buyer or end owner or end wearer of the article.

In a preferred embodiment of the present invention, the preparation 14 contains, as mentioned above, a buffer to help maintain the pH and stabilize pH drift. Any competent buffer can be used. Buffers are well known to those of ordinary skill in the art.

Still further, optionally, thickeners can be used in the preparation 14 to promote more even coating. Typical thickeners used preferably are non-greasy and non-oily compounds. Exemplary polymers and thickeners are listed in the CTFA Cosmetic Ingredient Handbook, 1 st Ed., J. M. Nikitakis ed., The Cosmetic, Toiletry and Fragrance Association, Washington, D.C. (1988) (hereafter CTFA Handbook), at pages 30, 47, 48, 67 and 97-100, incorporated herein by reference. Any thickeners that are well known to those skilled in the art can be used.

In some embodiments of the invention, the preparation 14 can include other optional ingredients, for example, antiperspirants and/or skin soothing substances, or the like. Skin soothing substances include, for example, skin moisturizing substances or skin anti-irritant substances. In addition, the preparation can also include other optional ingredients, for example, glycerin, which is a water-soluble emollient and emulsion aid, preservatives, fragrances, or dyes, or the like.

Examples of skin soothing substances include, for example, a skin moisturizing agent, especially for embodiments of the invention that are not dried onto the glove. Examples also include aloe vera, lotions, creams, and the like.

The acidic solution within the preparation typically includes an organic acid, such as a hydroxycarboxylic acid, herein termed a “hydroxy acid”. The acidic solution within the mixture typically includes an alpha-hydroxycarboxylic acid, herein termed an “alpha-hydroxy acid”. In accordance with an embodiment of the present invention, the acid solution present typically is a hydroxycarboxylic acid, generally an alpha-hydroxycarboxylic acid, for example, malic acid.

Exemplary hydroxy acids are disclosed in the incorporated-by-reference U.S. patent application Ser. No. 10/138,370. The particular amount of acid included in the preparation is dependent upon the type of acid, the production method and equipment, and the intended end use for the preparation-coated glove, for example, frequent or long-duration wearing, infrequent or short-duration wearing, use primarily to deter infection, or use to deter infection and also to exfoliate skin.

In one embodiment of the present invention, the preparation 14 contains about 0.1% to about 20% by weight of an acid, before being dry. Toward the higher end of this range, skin exfoliation abilities tends to be greater. In another embodiment of the present invention, the preparation contains about 0.1% to about 10% by weight of an acid, before being dry. In another embodiment of the present invention, the preparation contains about 0.2% to about 2% by weight of an acid, before being dry. The acid may be a hydroxy acid, or another type. Whatever the actual concentration or type of acid used, whether explicitly listed herein or not, the invention is preferably embodied so as also to achieve the earlier-discussed desired pH values.

Generally, cosmetologists and dermatologists use high concentrations of hydroxy acids (for example, 50 to 70 percent by weight) as superficial peels, to smooth rough skin, and to remove fine lines, acne scars, age spots, irregular pigmentation, and precancerous scaly patches. Moderate concentrations of hydroxy acids have typically been seen (for example, 10 to 50 percent by weight) to help control acne by unplugging pores, and to enhance the effectiveness of Retin-A and skin bleaches. However, at these concentrations, the hydroxy acid-containing products often provide dramatic results, but the potential to irritate or burn the skin is high. At hydroxy acid concentrations of, for example, 30% by weight or more, the compositions are capable of chemically burning the skin.

Accordingly, it is helpful to balance the acidic nature of an acidic solution with the skin-irritation potential of the solution. Many acid-containing compositions, including hydroxy acid-containing compositions, often warn the user that a tingling or burning sensation may be felt after the first several applications of the composition to the skin. In accordance with some embodiments of the present invention, it is preferable to provide an elastomeric flexible article, such as a disposable glove, that minimizes or avoids the tingling or burning sensation or irritation that can be associated with chemical burns due to acids, yet provide the beneficial antibacterial, anti-fungal, antiviral effects of these acids.

In accordance with an embodiment of the present invention, the hydroxy acid in the acidic solution may be any acid. For example, the hydroxy acid can be an aliphatic acid, e.g., glycolic acid; an aromatic acid, e.g., salicylic acid; or have aromatic and aliphatic components, e.g., mandelic acid. Exemplary hydroxy acids include the alpha-hydroxy acids, such as, but not limited to, glycolic acid, citric acid, lactic acid, tartaric acid, and malic acid. These alpha-hydroxy acids are naturally-occurring acids found in fruit, and have been used in skin care and skin treatment compositions for several years. It has been theorized that glycolic acid and lactic acid are the most effective alpha-hydroxy acids, if exfoliation is desired, because these acid molecules are small and more able to penetrate skin. Hydroxycaprylic acid is a synthetic alpha-hydroxy acid that has been used in skin care compositions. Other useful alpha-hydroxy acids are, for example, mandelic acid, leucic acid, azelaic, acid and ethylglycolic acid.

Beta-hydroxy acids, like salicylic acid, beta-hydroxypropionic acid and beta-hydroxybutyric acid, also are useful in the acidic solution of an embodiment of the present invention. In general, any aliphatic alpha- or beta-hydroxy acid having an aliphatic carbon chain containing two through ten carbon atoms can be used in the acidic solution. The hydroxy acid can be a monocarboxylic acid, a dicarboxylic acid, or a polycarboxylic acid.

The acid in the acidic solution is not limited to hydroxy acids. Essentially any acid that is used, or can be used, in cosmetic compositions for skin can be incorporated into the present solution. The acids traditionally are organic acids.

The acidic second layer of the disposable glove according to an embodiment of the present invention retains the characteristic of a disposable examination glove without any externally visible structural modification, and is easy and convenient to use. The affiliation between the acidic mixture (for example, a buffered malic acid solution) and the interior surface may be through a force provided by dehydration. Such affiliation is loosened when perspiration dissolves the dehydrated acidic pH solution. The longer a glove is worn, the more likely the hand will perspire, and consequently more acidic solution will be dissolved and disassociated from the glove surface, and be applied to the hand. The acidity of the solution can then condition hand skin and prevent microorganisms from growing under the wet condition.

In one embodiment, a solution of malic acid with a pH of about 5.5 is used to coat the gloves. Malic acid solution is distributed on the inner surface of the glove at a thickness of about 0.01 millimeter. Preferably, the distribution of the malic acid is substantially even and uniform. Preferably, the association between malic acid and the surface is achieved at least in part due to a non-covalent force provided through dehydration.

Further Discussion of Methodologies

Some embodiments of the present invention are methods for producing antimicrobial elastomeric flexible articles, for example, disposable gloves.

According to some of the method embodiments of the present invention, there is a method for producing gloves (or other article) having antimicrobial characteristic. According to one such method, a glove having at least two layers is to be formed, e.g., using a glove former, for example, a conventional glove former that is shaped somewhat like a hand. According to this method, a glove layer A (e.g., the inner “second” glove layer 12 discussed above) is formed or begins to be formed, e.g., on the glove former. Some time thereafter, another glove layer B (e.g., the outer “first” glove layer 10 discussed above) is formed over the glove layer A. For example, the layers A and B are laminated where the surfaces are in direct contact with each other. Thereafter, a coating A may be applied onto the available surface of the glove layer A (i.e., the surface that does not face the glove layer B), and a coating B may be applied onto the available surface of the glove layer B (i.e., the surface that does not face the glove layer A). For example, the coating B may be applied onto the glove layer B before the glove is stripped from the former, and the coating A may be applied onto the glove layer A after the glove is both stripped from the former and inverted such that the glove layer A faces outward. The glove layers A or B may be any combination of an outer and an inner layer, e.g., elastomeric layers, each as described anywhere in the present document or in the incorporated-by-reference documents. In addition to the specific materials discussed, any combination of suitable materials may be used, e.g., nitrile-nitrile, or any other combination. Similarly, the coatings A or B can each be any coating as described anywhere in the present document or in the incorporated-by-reference documents. In some specific example methods discussed in this document, the layer A and coating A are “outer”, relative to the layer B and coating B when the glove is to be worn. However, alternative implementations can form the layer A and coating A as “inner”, relative to the layer B and coating B when the glove is to be worn, for example, by including the re-ordering of some steps.

According to an embodiment of the invention, there is a method for producing gloves (or other articles) having antimicrobial characteristic. According to this method, gloves are to be formed on a formers, for example, conventional glove formers that are each shaped somewhat like a hand. According to this method, a glove is made as follows:

Preferably, a former is cleaned using any competent method, e.g., any conventional method;
preferably, the former is heated using any competent method, e.g., any conventional method;
a layer, which will be the outer layer when the glove is worn, is formed using any competent method, for example, any conventional method adapted to use a material composition that includes an amount of antimicrobial agent, preferably the amount by itself being an effective amount;
thereafter, a layer, which will be the inner layer when the glove is worn, is formed adjacent, preferably immediately adjacent, the outer layer using any competent method, for example, any conventional method adapted to use a material composition that does not include (or at least does not include as high a proportion of) the antimicrobial agent of the outer layer, wherein the inner layer shields the wearer's hand, during a period of wearing of the glove, from either or preferably both the antimicrobial agent on the outer layer and also any infectious agent that may have somehow penetrated the outer layer during wearing of the glove;
preferably, thereafter, an optional inner coating is applied onto the inner layer, onto what will be the inner surface of the inner layer when the glove is worn, either by spraying or by immersion, wherein the inner layer includes either or preferably both of a skin-soothing substance and an antimicrobial substance, this antimicrobial substance preferably being different from the antimicrobial substance in the outer layer and preferably being more suitable for prolonged exposure to skin than is the microbial substance in the outer layer, the antimicrobial substance for the optional inner coating preferably including an acid, preferably of a type that naturally-exists in a preferably edible plant and preferably including a buffer;
preferably, thereafter, the gloves are stripped from the formers and turned outside-out; and
preferably, thereafter, applying, preferably by spraying, an optional antimicrobial coating onto the outside surface of the outer layer.

### EXAMPLE METHODS

Some specific example implementations of methods according to some embodiments of the present invention are summarized below. Generally, the individual steps are self-explanatory, in view of previous description in this document and/or in the incorporated-by-reference documents U.S. Pat. No. 6,423,328 or 6,630,152 or U.S. patent application Ser. No. 10/138,370. A particular set of formulations that can be used in these example methods are summarized in some of Tables I-VIII.

### Example Method 1

Gloves (or other articles) having two layers are formed in which the outer layer is largely of natural rubber, the inner layer is largely of nitrile, and the optional outer and inner coatings are applied. As will be seen, chlorination is used to prepare the nitrile's inner surface. In this example method 1, as in the other example methods, not all steps are mandatory steps.

The example method 1 includes: dipping the former in powder-free coagulant, drying, dipping in natural rubber latex containing antimicrobial agent, drying/curing, leaching, drying, dipping in nitrile, drying/curing, leaching, chlorinating, leaching, drying/curing, dipping into inner coating mix, drying, stripping the glove from the former, drying/post-curing, surface treating (e.g., spray non-stick coating), spraying outer coating mix, drying.

Generally, the particular parameters for the example methods may be varied according to the requirements and wishes of the manufacturer. For example, different standards of dryness or leached-ness or vulcanization or cleanliness or the like may be sought to be reached by the manufacturer depending on the particular required quality or grade of the glove product.

One particular set of parameters, for the example method 1, is recited in this paragraph below within parentheses, in a re-listing of the example 1 method's steps. Variations of the parameters, and, more generally, determining of the parameters, will be within the expertise of those of ordinary skill in the art.

The example 1 method, re-listed: dipping the former in powder-free coagulant (e.g., the coagulant at about 45 degrees Celsius), drying (e.g., for about 3 minutes at about 65° C.), dipping in natural rubber latex containing antimicrobial agent (e.g., the latex at no more than about 40° C.), drying/curing (e.g., for about 20 minutes at about 110° C.), leaching (e.g., for about 3 minutes in warm water at about 85° C.), drying (e.g., for about 5 minutes at about 80° C.), dipping in nitrile (e.g., the nitrile at no more than about 40° C.), drying/curing (e.g., for about 20 minutes at about 20° C.), leaching (e.g., for about 3 minutes in water at about 85° C.), chlorinating (e.g., for about 2 minutes at about 350 parts per million at no more than about room temperature, e.g., followed by neutralization in, e.g., ammonia for, e.g., about 2 minutes), leaching (e.g., for about 5 minutes in cold water, e.g., at no more than about 40° C.), drying/curing (e.g., for about 15 minutes at about 85° C.), dipping into inner coating mix (e.g., the mixture at no more than about 40° C.), drying (e.g., for about 10 minutes at about 85° C.), stripping the glove from the former, drying/post-curing (e.g., by tumble drying for about 35 minutes at about 85° C. to 95° C.), surface treating (e.g., spray non-stick coating during the tumble drying using a conventional silicone-based coating), spraying outer coating mix (e.g., during the tumble drying), drying (e.g., by tumble drying for about 30 minutes at about 55° C. to 65° C.).

### Example Method 2

Gloves (or other articles) having two layers are formed in which the outer layer is largely of natural rubber, the inner layer is largely of nitrile, and the optional outer and inner coatings are applied. As will be seen, conventional polymer coating is used to prepare the nitrile's inner surface. The method includes (in imperative verb tense): dip the former in powder-free coagulant, dry, dip in natural rubber latex containing antimicrobial agent, dry/cure, leach, dry, dip in nitrile, dry/cure, leach, dry, coat with polymer, dry, leach, dry, apply inner coating mix, dry, strip, dry, surface treat (e.g., spray non-stick coating), spray outer coating mix, and dry.

### Example Method 3

Gloves (or other articles) having two layers re formed in which the outer layer is largely of natural rubber, the inner layer is largely of nitrile, and the optional outer and inner coatings are applied. As will be seen, chlorination is used to prepare the nitrile's inner surface, and both the outer and inner coatings are applied after the gloves are stripped from the formers. The method includes: dipping the former in coagulant, drying, dipping in natural rubber latex containing antimicrobial agent, drying/curing, leaching, drying, dipping in nitrile, drying/curing, leaching, chlorination, leaching, drying, stripping, chlorination, rinsing (e.g., in warm water), rinsing again (e.g., in cold water), dipping in inner coating solution, drying, spray outer coating, and drying. Note that the dipping in inner coating solution, after stripping has already occurred, can coat both the inner and outer coatings with the inner coating solution, whereas spraying the outer coating can be expected to coat substantially only the outer coating.

### Example Method 4

Gloves (or other articles) having two layers re formed in which the outer layer is largely of nitrile, the inner layer is largely of polyurethane, and the optional outer and inner coatings are applied. The method includes: dipping the former in powder-free coagulant, drying, dipping in nitrile containing antimicrobial agent, drying/curing, leaching, drying, dipping in polyurethane, leaching, drying, dipping in inner coating mix, drying, stripping, drying, surface treating (spraying non-stick coating), drying, spraying outer coating, and drying.

### Example Method 5

Gloves (or other articles) having two layers re formed in which the outer layer is largely of nitrile, the inner layer is largely of polyurethane, and the optional outer and inner coatings are applied. As will be seen, both the outer and inner coatings are applied after the gloves are stripped from the formers. The method includes: dipping the former in coagulant, drying, dipping in nitrile containing antimicrobial agent, drying/curing, leaching, drying, polyurethane coating, leaching, drying, stripping, rinsing, rinsing again, dipping in inner coating mix, drying, surface treating (spraying non-stick coating), spraying outer coating, and drying.

### Example Method 6

Gloves (or other articles) having two layers re formed in which the outer layer is largely of polyvinyl chloride (PVC), the inner layer is largely of polyurethane, and the optional outer and inner coatings are applied. The method includes: dipping in PVC containing antimicrobial agent, drying, polyurethane coating, drying, dipping in inner coating solution, drying, stripping, drying, spraying outer coating, drying.

### Example Method 7

Gloves (or other articles) having two layers re formed in which the outer layer is largely of PVC, the inner layer is largely of polyurethane, and the optional outer and inner coatings are applied. As will be seen, both the outer and inner coatings are applied after the gloves are stripped from the formers. The method includes: dipping in PVC containing antimicrobial agent, drying, polyurethane coating, drying, stripping, dipping in inner coating solution, drying, spray outer coating, drying.

Example methods and articles according to some embodiments of the present invention have already been described. A particular set of example formulations that might be used with the example methods 1-7 are shown in Tables A-G. The example formulations are merely examples, and it would be apparent to those of ordinary skill in the art that the example formulations can be changed, even dramatically in some cases, and still embody various embodiments of the present invention. For example, many ingredients in the tables are merely optional ingredients or particular embodiments or examples of classes of ingredients. Merely for example, vulcanization accelerator, or antioxidant, or colorant, or the like are optional ingredients. For another example, the example quantities or quantity ranges are merely examples according to particular embodiment(s) of the invention; in other embodiments, the quantities or quantity ranges can vary. In the various tables, the symbol “˜” is used to mean “about”; thus, “˜5” would mean “about 5”, for example.

TABLE A
Example antimicrobial composition for adding into glove-layer
material (e.g., latex or nitrile compounding materials)

| | Example actual or active parts |
|---|---|
| Example dispersion ingredient | by weight |
| Triclosan (e.g., content ≧ ˜99%) | ˜5 to ˜20 |
| Potassium Oleic(e.g., content ≧ ˜99%) | ˜5 to ˜20 |
| (Soft) Water | ˜60 to ˜90 |

Example preparation details
Raising the temperature of the mixture to ˜60° C.-˜80° C., emulsify the mixture in high speed for 20 minutes until the mixture becomes stable emulsion

TABLE B
Example formulation for an (exterior-side) antimicrobial
coating (e.g., spray preparation)

| | Example actual or active parts |
|---|---|
| Example dispersion ingredient | by weight |
| Triclosan (e.g., content ≧ ˜99%) | ˜2 to ˜50 |
| Alcohol (e.g., content ≧ ˜99%) | ˜50 to ˜98 |

Example preparation/usage details
Mix until the triclosan dissolves. For example, for every 1,000 grams of gloves, use, e.g., 10-500 grams of the spraying solution. For example, every 100 grams of gloves might contain, e.g., 0.1-5 gram of triclosan.

TABLE C
Example Latex Composition

| | Example | | Example |
|---|---|---|---|
| | actual dry or | | actual |
| Example dispersion | active parts | Example | parts by |
| ingredient | by weight | concentration | weight |
| Natural Rubber Latex | ˜100 | ˜60% | ˜167 |
| Potassium Hydroxide | ˜0.2 to ˜2 | ˜20% | ˜1 to ˜10 |
| Casein | ˜0.2 to ˜2 | ˜10% | ˜2 to ˜20 |
| Zinc Oxide | ˜0.2 to ˜2 | ˜50% | ˜0.4 to ˜4 |
| Vulcanization Accelerator | | | |
| Zinc Diethyl | ˜0.2 to ˜2 | ˜50% | ˜0.4 to ˜4 |
| Dithiocarbamate | | | |
| Antioxidant | | | |
| Wingstay ® L | ˜0.2 to ˜2 | ˜50% | ˜0.4 to ˜4 |
| 2,6-Di-tert-butyl-4-methyl | ˜0.2 to ˜2 | ˜50% | ˜0.4 to ˜4 |
| phenol | | | |
| Sulphur | ˜0.2 to ˜2 | ˜50% | ˜0.4 to ˜4 |
| Colorant | ˜0.2 to ˜2 | ˜50% | ˜0.4 to ˜4 |
| Antimicrobial Composition | ˜0.2 to ˜2 | ˜10% | ˜2 to ˜20 |

TABLE D
Example Nitrile Composition
(e.g., for example processes 1, 2, 3)

| | Example | | Example |
|---|---|---|---|
| | actual dry or | | actual |
| Example dispersion | active parts | Example | parts by |
| ingredient | by weight | concentration | weight |
| Nitrile | ˜100 | ˜43% | ˜233 |
| Potassium Hydroxide | ˜0.2 to ˜2 | ˜20% | ˜1 to ˜10 |
| Sulphur | ˜0.2 to ˜2 | ˜50% | ˜0.4 to ˜4 |
| Zinc Oxide | ˜0.2 to ˜2 | ˜50% | ˜0.4 to ˜4 |
| Vulcanization Accelerator | | | |
| Zinc Diethyl | ˜0.2 to ˜2 | ˜50% | ˜0.4 to ˜4 |
| Dithiocarbamate | | | |
| Antioxidant | | | |
| 2,6-Di-tert-butyl-4-methyl | ˜0.2 to ˜2 | ˜50% | ˜0.4 to ˜4 |
| phenol | | | |
| Colorant | ˜0.2 to ˜2 | ˜50% | ˜0.4 to ˜4 |

TABLE E
Example Nitrile Composition (e.g., for example processes 4, 5)

| | Example | | Example |
|---|---|---|---|
| | actual dry or | | actual |
| Example dispersion | active parts | Example | parts by |
| ingredient | by weight | concentration | weight |
| Nitrile | ˜100 | ˜43% | ˜233 |
| Potassium Hydroxide | ˜0.2 to ˜2 | ˜20% | ˜1 to ˜10 |
| Sulphur | ˜0.2 to ˜2 | ˜50% | ˜0.4 to ˜4 |
| Zinc Oxide | ˜0.2 to ˜2 | ˜50% | ˜0.4 to ˜4 |
| Vulcanization Accelerator | | | |
| Zinc Diethyl | ˜0.2 to ˜2 | ˜50% | ˜0.4 to ˜4 |
| Dithiocarbamate | | | |
| Antioxidant | | | |
| 2,6-Di-tert-butyl-4-methyl | ˜0.2 to ˜2 | ˜50% | ˜0.4 to ˜4 |
| phenol | | | |
| Colorant | ˜0.2 to ˜2 | ˜50% | ˜0.4 to ˜4 |
| Antimicrobial Composition | ˜0.2 to ˜2 | ˜10% | ˜2 to ˜20 |

TABLE F1
Example formulation for an (interior-side) antimicrobial coating
(e.g., immersion preparation)

| | Example dry or active parts by |
|---|---|
| Example dispersion ingredient | weight |
| Citric Acid | ˜0.2 to ˜5 |
| Sodium Citrate | ˜0.2 to ˜5 |
| carboxymethyl cellulose (CMC) | ˜0.01 to ˜1 |
| Aloe Powder | ˜0.01 to ˜1 |
| Sodium Benzyl | ˜0.01 to ˜1 |
| Potassium Sorbate | ˜0.01 to ˜1 |
| (Soft) Water | ˜86 to ˜99.56 |

TABLE F2
Example formulation for an (interior-side) antimicrobial coating
(e.g., immersion preparation)

| | Example dry or active parts by |
|---|---|
| Example dispersion ingredient | weight |
| Benzyl Acid | ˜0.2 to ˜5 |
| Sodium Malic | ˜0.2 to ˜5 |
| CMC | ˜0.01 to ˜1 |
| Aloe Powder | ˜0.01 to ˜1 |
| Sodium Benzyl | ˜0.01 to ˜1 |
| Potassium Sorbate | ˜0.01 to ˜1 |
| (Soft) Water | ˜86 to ˜99.56 |

TABLE G
Example Vinyl Composition

| | Example actual dry or active |
|---|---|
| Example dispersion ingredient | parts by weight |
| PVC | ˜100 |
| dioctyl phthalate (DOP) | ˜70-˜85 |
| 2,2,4-Trimethyl-1,3-pentanediol | ˜5 to ˜20 |
| diisobutyrate (TXIB) | |
| Stabilizers | ˜1 to ˜5 |
| Viscosity Reducing Agent | ˜5 to ˜30 |
| Antimicrobial Composition | ˜0.2 to ˜2 |

| | Example | Example actual |
|---|---|---|
| Example dispersion ingredient | concentration | parts by weight |
| Polyurethane | ˜30% | ˜1 |
| (Soft) Water | | ˜9 to ˜15 |

The example methods 1-7 have been discussed. Of course, other specific implementations of methods are possible. In the above example methods, the main/outer layers contain antimicrobial ingredient in their mixture. To compensate for the loss of the antimicrobial ingredient during the leaching/manufacturing process, the finished glove can have an additional antimicrobial coating sprayed onto the outer surface. The inner surface has an inner antimicrobial coating that is preferably different from the outer antimicrobial coating. For example, the inner coating may be an acidic coating that is buffered at or near the skin's natural pH to inhibit bacterial growth. In order to protect the user's skin from coming to direct contact with other outer layers that contain the antimicrobial agent (to avoid the bacteria resistance issue, and for latex outer layers also to avoid the protein sensitivity issue), there is an inner layer that blocks the main/outer layer. The inner layer does not have nearly as high a proportion of substances (e.g., the outer layer's antimicrobial agent or latex proteins) that may be harmful in contact (or prolonged contact) with skin. The inner layer shields the wearer's skin from the outer layer.

Further, in addition to the specific preparations discussed in the present document, or an Aloe Vera solution as discussed in the two references incorporated above (U.S. Pat. No. 6,274,154 or U.S. patent Ser. No. 09/938,715).

Again, any embodiment of the present invention may be embodied to alternatively or additionally use any other substance (e.g., any preparation) that can be dried or otherwise applied onto the inside of a glove and that, in the inside of the glove during wearing, is beneficial to the hand. Further the any other substance preferably does not require moisture to be artificially introduced into the glove after donning; instead, the only moisture to be introduced into the inner surface of the glove after donning is from perspiration from a hand during wearing of the glove.

Throughout the description and drawings, example embodiments, for example, products and methods, are given with reference to specific embodiments and configurations. However, the present invention is not limited to those specific embodiments or configurations. It will be appreciated by those of ordinary skill in the art that the present invention can be embodied in other specific forms without departing from the spirit and scope of the present invention.

For example, although glove embodiments are illustrated in FIGS. 1 and 2, any other article or form that contacts skin may also embody the present invention. For example, the present invention may be embodied as elastomeric flexible peels, articles, wraps, and (other) medical devices. Similarly, the composition and application of the preparation may be varied without departing from the spirit and scope of the present invention. For example, various different preparations may be utilized to obtain an ultimate final antimicrobial elastomeric flexible article, for example, a glove, that has characteristics as described within the present document. For example, the formulations of the preparation may be varied in order to have a thicker or thinner coating or layers, as desired to control comfort in use, dexterity, sense of feel, or protection. Still other changes would be apparent.

The scope of the invention is not limited merely to the specific example embodiments or configurations of the foregoing description, but rather is indicated by the appended claims. All changes that come within the meaning and range of equivalents within the claims are intended to be understood as being embraced within the scope of the claims.

1. A disposable protective glove comprising:
a first layer, with an effective amount of antimicrobial agent therein or thereon; and
a second layer, to be closer to a hand than the first layer, when the glove is worn on the hand, the second layer configured to resist, when the glove is worn, penetration by the anti-microbial agent and thereby to resist contact between the anti-microbial agent with the hand.

2. A glove according to claim 1, wherein the anti-microbial agent is selected from the group consisting of halogenated hydroxy diphenyl derivatives such as diphenyl ethers, phenol derivatives, diacetylamino-azotoluene and triclorocarban, 2,4,4′trichloro-2′-hydroxydiphenyl ether, chlorophene, and dichloroxylenol, hexachlorophane.

3. A glove according to claim 2, wherein the antimicrobial agent comprises 2,4,4′trichloro-2′-hydroxydiphenyl ether.

4. A glove according to claim 3, wherein the glove contains from 0.1 to 10% by weight of 2,4,4′trichloro-2′-hydroxydiphenyl ether.

5. A glove according to claim 4, wherein the glove contains from 0.1 to 5% by weight of 2,4,4′trichloro-2′-hydroxydiphenyl ether.

6. A glove according to claim 5, wherein the glove contains from about 0.3 to 3% by weight of 2,4,4′trichloro-2′-hydroxydiphenyl ether.

7. A glove according to claim 1, wherein:
the second layer includes an interior surface;
the glove further comprises a preparation disposed on the interior surface;
the preparation includes an additional anti-microbial substance; and
the preparation includes a buffer that helps resist change in pH during wearing of the disposable protective glove.

8. A glove according to claim 7, wherein the additional antimicrobial substance is acidic during a period when the protective glove is worn, and wherein acidity of the additional antimicrobial substance contributes substantially to anti-microbial properties of the antimicrobial substance.

9. A glove according to claim 8, wherein the preparation has pH within a range of 4.5 to 6.0 during a period in which the preparation is moist.

10. A glove according to claim 7, wherein the additional antimicrobial substance includes an acid that exists naturally in an edible plant.

11. A glove according to claim 7, wherein the preparation further includes a skin soothing substance.

12. A glove according to claim 11, wherein the skin soothing substance includes dehydrated aloe vera.

13. A glove according to claim 1, wherein the first layer is made from a material selected from the group consisting of a resinous material and a polymer material.

14. A glove according to claim 1, wherein the first layer is made of a single layer selected from the group consisting of natural rubber latex, acrylonitrile, vinyl, chloroprene, and polyvinyl chloride.

15. A glove according to claim 1, wherein the second layer is made of a single layer of fluid-impermeable material.

16. A glove according to claim 1, wherein the antimicrobial agent is evenly distributed within the first layer of the protective glove.

17. A glove according to claim 1, wherein the antimicrobial agent is evenly dispersed on the outer surface of the first layer.

18. A disposable protective article comprising:
an outer layer having an antimicrobial agent distributed within or applied onto the outer layer; and
an inner layer to be closer to the skin than the outer layer, the inner layer having less proteins than natural rubber latex and comprising an interior surface with a skin conditioning or soothing substance dispersed thereon;
wherein the inner layer serves as a barrier between the skin and the outer layer so to resist developing of antimicrobial resistance in microbes on the skin and some of the skin conditioning or soothing substance will interact physically with perspiration from the skin and due thereto increase in ability to condition or soothe the skin.

19. A disposable protective article according to claim 18, wherein the antimicrobial agent is distributed within the outer layer and applied onto the outer layer.

20. A disposable protective article according to claim 18, wherein the skin conditioning or soothing substance is a dehydrated preparation.

21. A disposable protective article according to claim 18, wherein the skin conditioning or soothing substance also contains an additional antimicrobial agent.

22. A disposable protective article according to claim 18, wherein the inner layer comprises polyurethane, chloroprene or polymer.

23. A disposable protective article according to claim 18, wherein the disposable protective article comprises a disposable protective examination glove.

24. A method for making a disposable protective article for protecting skin, the disposable protective article to comprise multiple layers, the method comprising:
forming a first layer, the first layer comprising a material that includes an antimicrobial agent dispersed within;
forming a second layer, the second layer to be closer to the skin than the first layer when the disposable protective article is in use, wherein the second layer is to help resist contact between skin and the antimicrobial agent when the disposable protective article is used on skin.

25. A method according to claim 24, wherein the step of forming the first layer precedes the step of forming the second layer.

26. A method according to claim 24, wherein the antimicrobial agent is hereinafter referred to as zeroth antimicrobial agent, the method further comprising:
applying a first preparation to a surface of the first layer, the first preparation including a first antimicrobial agent.

27. A method according to claim 26, the method further comprising:
applying a second preparation to a surface of the second layer, the second preparation including a skin conditioning substance that is activated by perspiration from the hand.

28. A method according to claim 26, the method further comprising:
applying a second preparation to a surface of the second layer, the second preparation including a second antimicrobial agent, the second antimicrobial agent being different in kind from the zeroth antimicrobial agent.

29. A method according to claim 24, wherein the antimicrobial agent is hereinafter referred to as zeroth antimicrobial agent, the method further comprising:
applying a preparation to a surface of the second layer, the preparation including:
an antimicrobial agent different in kind from the zeroth antimicrobial agent, a skin conditioning substance, or
a skin conditioning substance.

30. A method according to claim 24 wherein the disposable protective article comprises a disposable protective glove.

31. A method according to claim 24 wherein the step of forming a first layer comprises coating a former with a composition that includes latex, nitrile, or PVC.
